(19) United States
(12) Patent Application Publication
(10) Pub. No.: US 2025/0135067 A1
(43) Pub. Date: May 1, 2025

(54) PLATE ASSEMBLY FOR TISSUE COMPRESSION SYSTEM AND CORRESPONDING METHOD

(71) Applicant: Arthrex, Inc., Naples, FL (US)

(72) Inventors: Eric Zajac, Naples, FL (US); Thomas Dooney, JR., Naples, FL (US); Andrew Christian Petry, Naples, FL (US); Jennifer Brooks, Naples, FL (US); John Tokish, Scottsdale, AZ (US)

(21) Appl. No.: 18/384,435

(22) Filed: Oct. 27, 2023

(51) Int. Cl. A61L 27/36

(52) U.S. Cl. CPC A61L 27/3691

(57) ABSTRACT

This disclosure details a plate assembly for use with a tissue compression system and corresponding methods. In an example system, the system includes include a press and a plate assembly. The plate assembly includes a first plate defining a cavity configured to receive harvested tissue and a second plate including a projection insertable into the cavity. The press is configured to apply pressure to the plate assembly to compress the harvested tissue. The plate assembly is configured to hold the harvested tissue under compression when the plate assembly is removed from the press.

### BACKGROUND

To re-establish stability within a shoulder joint after injury, for example, autograft tissue can be employed to span the humerus and the glenoid cavity of the scapula and repair the rotator cuff. Allograft tissue is an alternate option.

### SUMMARY

This disclosure details a plate assembly for use with a tissue compression system and corresponding methods. The tissue compression system is configured to change one or more dimensions of harvested tissue, such as autograft, which is harvested from one location in a patient's body and used for a surgical repair or reconstruction procedure in another location in the patient's body. This disclosure also relates to the method of changing one or more dimensions of the harvested tissue, as well as the method of performing a surgical procedure including one or more steps associated with changing the dimension(s) of the harvested tissue.

Among other benefits, the disclosed plate assembly permits a user to hold the graft under compression after removing the plate assembly from a press. Doing so maintains the shape of the graft after removal from the press. In one particular aspect, the plate assembly allows the user can to apply suture to the graft while the graft is held under compression by the plate assembly.

In some aspects, the techniques described herein relate to a system, including: a press; and a plate assembly including a first plate defining a cavity configured to receive harvested tissue and a second plate including a projection insertable into the cavity, wherein the press is configured to apply pressure to the plate assembly to compress the harvested tissue, and wherein the plate assembly is configured to hold the harvested tissue under compression when the plate assembly is removed from the press.

In some aspects, the techniques described herein relate to a surgical method, including: placing harvested tissue into a cavity of a first plate; arranging a second plate relative to the first plate such that a projection of the second plate is configured to fit within the cavity of the first plate; arranging the first plate, second plate, and harvested tissue relative to a press; changing a dimension of the harvested tissue by using the press to apply pressure to the first and second plate; and removing the first plate, second plate, and harvested tissue from the press with the first and second plate held together such that the first and second plate hold the harvested tissue under compression.

### BRIEF DESCRIPTION OF THE DRAWINGS

FIG. 1 is a perspective view of an example press.

FIG. 2 is a perspective view of an assembly configured for use with the press. The assembly includes a first press component, a second press component, a first plate, and a second plate.

FIG. 3 is a top perspective view of the first press component.

FIG. 4 is a bottom perspective view of the second press component.

FIG. 5 is a top perspective view of the first plate.

FIG. 6 is a bottom perspective view of the second plate.

FIG. 7 is a cross-sectional view of a portion of an opening in the first plate, taken along line 7-7 from FIG. 5.

FIG. 8 is a top view of the second plate.

FIG. 9 is a view of the first plate spaced-apart from the second plate such that a piece of harvested tissue can be placed into the cavity of the first plate.

FIG. 10 illustrates the first plate and second plate in a condition in which posts of the second plate project into, and through, openings in the first plate.

FIG. 11 is a view similar to FIG. 9, but with the projection of the second plate being partially inserted into the cavity such that the piece of harvested tissue is compressed relative to its state in FIG. 9.

FIG. 12 is a view representative of a user threading suture relative to a piece of harvested tissue, while the harvested tissue is held under compression between the first and second plates.

### DETAILED DESCRIPTION

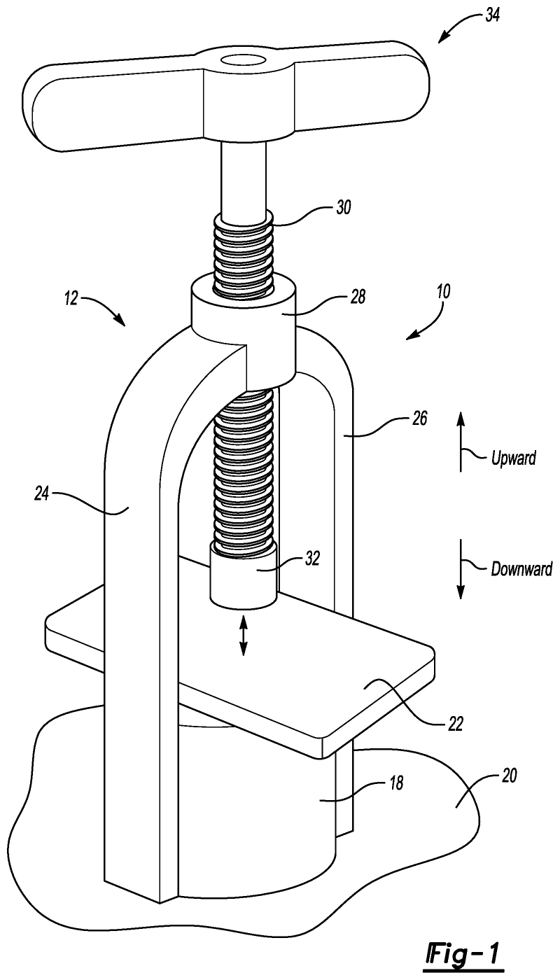

This disclosure details a plate assembly for use with a tissue compression system and corresponding methods. With reference to the drawings, FIG. 1 illustrates a portion of an example surgical system 10 (“system 10”), which may be referred to as a tissue compression system. As shown in FIG. 1, the system 10 includes a press 12. The press 12, in this example, is manually operated and is configured to apply pressure to structures arranged relative to the press 12. Specifically, some components of the press 12 are configured to move to compress, and thereby flatten and change one or more dimensions of, a harvested piece of tissue. While the press 12 is manually operated in this example, one or more components of the press 12 could be controlled using an electromechanical and/or hydraulic device, such as an actuator. Further, while an example press 12 is shown in FIG. 1, it should be understood that this disclosure extends to other devices configured to apply pressure to other structures, including including clamps, vises, levers, etc.

In this example, the press 12 includes a stand 18 configured to rest on a worktop 20 or other surface, such as a table. The press 12 also includes a base 22 mounted above the stand 18. With reference to directional terms, the “upward” and “downward” directions are labeled in some drawings and are used with reference to the normal operating orientation of the associated components. In this example, the base 22 remains stationary during use of the press 12.

The press 12 further includes a pair of support arms 24, 26 projecting from opposite sides of the stand 18 and converging on a nut 28 spaced upward of the base 22. The nut 28 is configured to remain stationary during use of the press 12. The nut 28 includes a threaded through-bore which receives a shaft 30. The shaft 30 is at least partially threaded, and the threads of the shaft 30 cooperate with threads of the nut 28 such that rotation of the shaft 30 results in movement of the shaft 30 in either the upward or downward direction relative to the nut 28 depending on the direction of rotation.

Below the nut 28, the shaft 30 is connected to a connector 32, which is mounted adjacent an end of the shaft 30. The connector 32 is configured to transmit vertical movement of the shaft 30 to a structure on the base 22, for example. The connector 32 is mounted to the shaft 30 such that rotational movement of the shaft 30 is not transmitted to the connector 32. The connector 32 can be integrally formed with the shaft 30 in other examples.

Above the nut 28, the shaft 30 is connected to a handle 34. The handle 34 permits a user, such as a surgeon or surgical assistant, to apply a rotational force to the shaft 30 via the handle 34. Ultimately, the user is able to selectively raise and lower the connector 32 by rotating the handle 34.

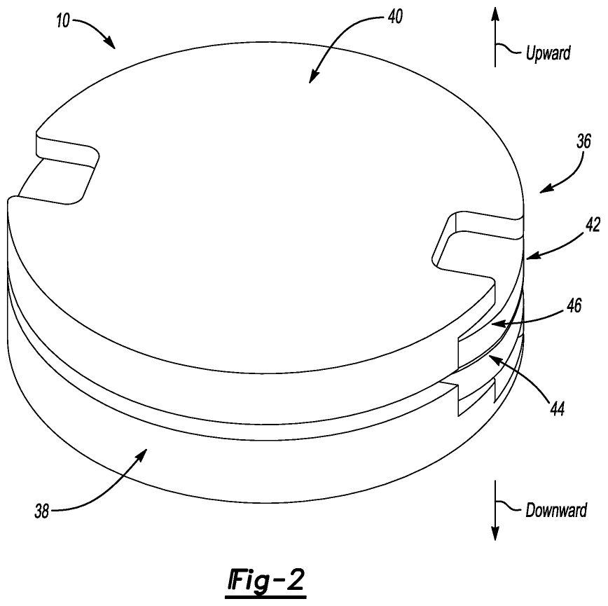

The surgical system also includes an assembly 36, as shown in FIG. 2. The assembly 36 is configured to receive harvested tissue and to be positioned relative to the press 12 such that the press 12 can apply pressure to the assembly 36. The assembly 36 is configured to transfer pressure from the press 12 to the harvested tissue such that the harvested tissue is flattened.

In this example, the assembly 36 includes a first press component 38 configured to rest on the base 22. The assembly 36 further includes a second press component 40 configured to directly contact the connector 32. Vertically between the first and second press components 38, 40, the assembly 36 includes a plate assembly 42, which includes a first plate 44 and a second plate 46. The first plate 44 is arranged adjacent the first press component 38 and is configured to be held in place by the first press component 38. The second plate 46 is arranged vertically above the first plate 44.

With respect to the term “component,” as used to refer to the first press component 38 and the second press component 40 specifically, the term “component” is not intended to be a nonce term or placeholder serving as a substitute for “means.” Rather, the term refers to the first and second press components 38, 40 being structures that facilitate use of the assembly 36 relative to press 12. The first and second press component 38, 40 may also be referred to as plates.

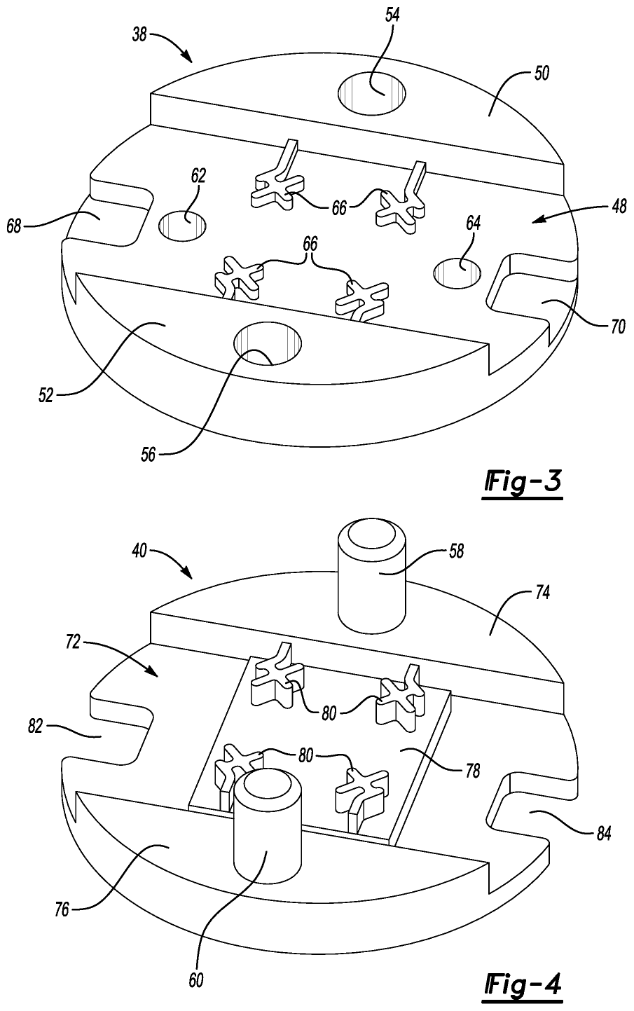

FIG. 3 illustrates the first press component 38 from a top perspective view. As shown, the first press component 38 is substantially circular. The first press component 38 exhibits a channel 48 extending across an entire diameter of the first press component 38, in this example. On opposite sides of the channel 48, the first press component 38 exhibits side sections 50, 52. Within the side sections 50, 52, the first press component 38 exhibits a greater height dimension than within the channel 48. The height of the first press component 38 is measured in the upward and downward directions (i.e., generally in-and-out of the page relative to FIG. 3), relative to a bottom surface of the first press component 38, which is substantially flat in this example, and may include one or more features configured to facilitate attachment of the first press component 38 to the base 22.

The side sections 50, 52 each include an opening 54, 56 configured to receive a corresponding guide pin 58, 60 (FIG. 4) of the second press component 40. The openings 54, 56 could be through-bores extending fully through the side sections 50, 52, or may extend only partially through the height of the side sections 50, 52. Within the channel 48, the first press component 38 includes first and second openings 62, 64 configured to receive posts of the second plate 46, as discussed below. The first and second openings 62, 64 are spaced-outward (i.e., relative to a center of the first press component) of four projections 66, which are configured to be received within slots of the first plate 44, as discussed below. Outward of the first and second openings 62, 64, and at ends of the channel 48, the first press component 38 further includes recessed sections 68, 70. Within the recessed sections 68, 70, the first press component 38 exhibits a lesser height than within the channel 48. The recessed sections 68, 70 open to an outer perimeter of the first press component 38.

FIG. 4 illustrates the second press component 40 from a bottom perspective view. As shown, the second press component 40 is substantially circular, and in particular exhibits a diameter substantially equal to the first press component 38. The first and second press components 38, 40 need not be circular in all embodiments. The second press component 40 exhibits a channel 72 extending across an entire diameter of the second press component 40, in this example. On opposite sides of the channel 72, the second press component 40 exhibits side sections 74, 76. Within the side sections 74, 76, the second press component 40 exhibits a greater height dimension than within the channel 72. The height of the second press component 40 is measured in the upward and downward directions relative to a top surface of the second press component 40, which may be flat or may include one or more attachment features configured to facilitate attachment to the connector 32. The side sections 74, 76 each include a corresponding guide pin 58, 60, which are configured to project into a corresponding opening 54, 56 in the first press component 38.

Between the side sections 74, 76, the second press component 40 includes a projection, or raised section, 78, within which the second press component 40 exhibits a greater height than in the channel 72. The raised section 78 is configured to be received in a recess in the second plate 46. The raised section 78 includes four projections 80, which are configured to be received within slots of the second plate 46, as discussed below. At ends of the channel 72, the second press component 40 includes notches 82, 84, which are open to an outer perimeter of the second press component 40, and which align vertically with the recessed sections 68, 70 of the first press component 38 when the guide pins 58, 60 are received within the openings 54, 56.

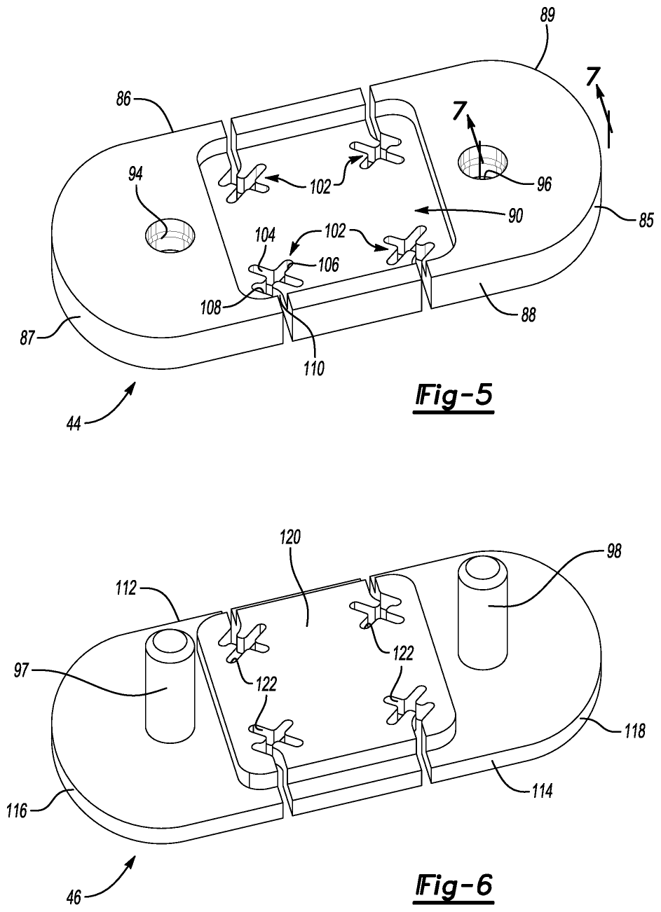

FIG. 5 illustrates the first plate 44 from a top perspective view. The first plate 44 includes an outer perimeter defined by first and second sides 86, 88, which extend substantially parallel to one another. The first and second sides 86, 88 are connected together by arcuate sides 87, 89, which are curved and correspond to a curvature of the first and second press component 38, 40, in this example. The first and second sides 86, 88 are spaced-apart from one another by a distance substantially equal to, but slightly less than, the distance between side sections 50, 52, such that the first plate 44 can be placed into the channel 48 with the first and second sides 86, 88 adjacent a corresponding one of the side sections 50, 52.

The first plate 44 includes a cavity 90 configured to receive a piece of harvested tissue. The cavity 90 exhibits a lesser height than the surrounding portions of first plate 44, measured generally in the upward and downward directions relative to a bottom surface of the first plate 44, which is substantially flat in one example. The cavity 90 exhibits a substantially rectangular perimeter, with rounded corners, in this example. Outward of the cavity, the first plate 44 includes first and second openings 94, 96 configured to receive corresponding posts 97, 98 (FIG. 6) of the second plate 46. The openings 94, 96 are through holes extending through the entire height of the first plate 44, in this example. The openings 94, 96 are configured to create friction with the posts 97, 98 to resist removal of the posts 97, 98 relative to the openings 94, 96. In one example, the openings 94, 96 include an elastomeric liner, such as an elastomeric O-ring, configured to resist removal of the posts 97, 98 from the openings 94, 96. An example O-ring 100 is shown relative to the opening 96 in FIG. 7. In other examples, the openings 94, 96 may resist removal of the posts 97, 98 without an elastomeric liner, such as by being dimensioned to provide a friction fit with the posts 97, 98.

The first plate 44 further includes four slots 102. The slots 102 each extend through an entirety of a height of the first plate 44. The slots 102 each extend from the outer perimeter of the first plate 44 to the cavity 90. The slots 102 each include, in this example, a plurality of substantially straight sections arranged relative to one another, and intersecting one another, to form a shape that facilitates passing suture relative to a piece of harvested tissue, as will be discussed below.

With reference to one of the slots 102, the slot 102 includes a first section 104 and a second section 106 extending substantially perpendicular to and intersecting with the first section 104. The first and second sections 104, 106 form a substantial X-shape adjacent a corner of the cavity 90. An end of the second section 106 proximate an adjacent corner of the cavity 90 terminates into a third section 108. The third section 108 is shorter than the first and second sections 104, 106. The sections, especially sections 104, 106, 108, are sized so as to permit one to thread suture relative to differently-sized pieces of harvested tissue, and to form various stitches relative to the harvested tissue. An end of the third section 108 proximate the side 88 terminates into a fourth section 110, which extends substantially perpendicular to the side 88. The fourth section 110 opens to the perimeter of the first plate 44 at the side 88. While one slot 102 has been described, the remaining slots are configured in substantially the same manner.

FIG. 6 illustrates the second plate 46 from a bottom perspective view. The second plate 46 includes an outer perimeter defined by first and second sides 112, 114, which extend substantially parallel to one another. The first and second sides 112, 114 are connected together by arcuate sides 116, 118, which are curved and correspond to a curvature of the first and second press component 38, 40, in this example. Within the perimeter, the second plate 46 includes a projection 120 sized and shaped substantially equal to, but slightly less than, the dimensions of the cavity 90 such that the projection 120 can project into the cavity 90 to contact and apply pressure to harvested tissue.

The projection 120 includes four slots 122. The slots 122 each extend through an entirety of a height of the second plate 46. The slots 122 further extend from the outer perimeter of the second plate 46 to the projection 120. The slots 122 are sized and arranged to substantially match a corresponding one of the slots 102 of the first plate 44. When the posts 97, 98 of the second plate 46 are received in openings 94, 96 of the first plate, the slots 122 vertically overlap a corresponding one of the slots 102. In other words, the slots 102, 122 are vertically spaced-apart from one another but vertically overlap one another, and aligned in the direction of the length and width of the first and second plates 44, 46.

While there are four of each of the slots 102, 122, and four of each of the projections 66, 80, this disclosure extends to embodiments with a different number of slots and projections.

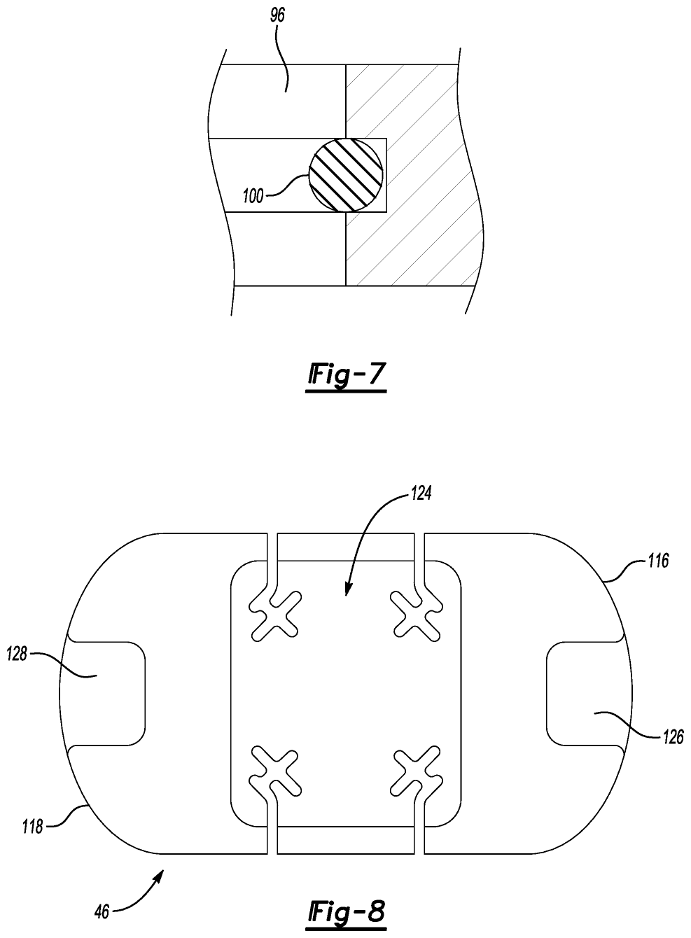

FIG. 8 illustrates the second plate 46 from a top view. As shown, opposite the projection 120, the second plate 46 includes a recessed section 124 configured to receive raised section 78 of the second press component 40. Further, adjacent the arcuate sides 116, 118, the second plate 46 includes first and second recessed sections 126, 128 open toward the outer perimeter of the second plate 46. The first and second recessed section 126, 128 are each configured to interface with a clamp, for example. The first plate 44 may include corresponding first and second recessed sections in a bottom surface thereof. Recessed sections 68, 70 and notches 82, 84 of the first and second press components 38, 40 facilitate attachment of one or more clamps, or similar devices, to the first and second plates via the first and second recessed sections 126, 128, and any corresponding recessed sections of the first plate 44.

An example method of use will now be described. A surgeon may perform the method either partially or entirely. One or more steps of the method may be performed by another, such as a surgical assistant. The method may be performed either partially or entirely during an arthroscopic surgical procedure.

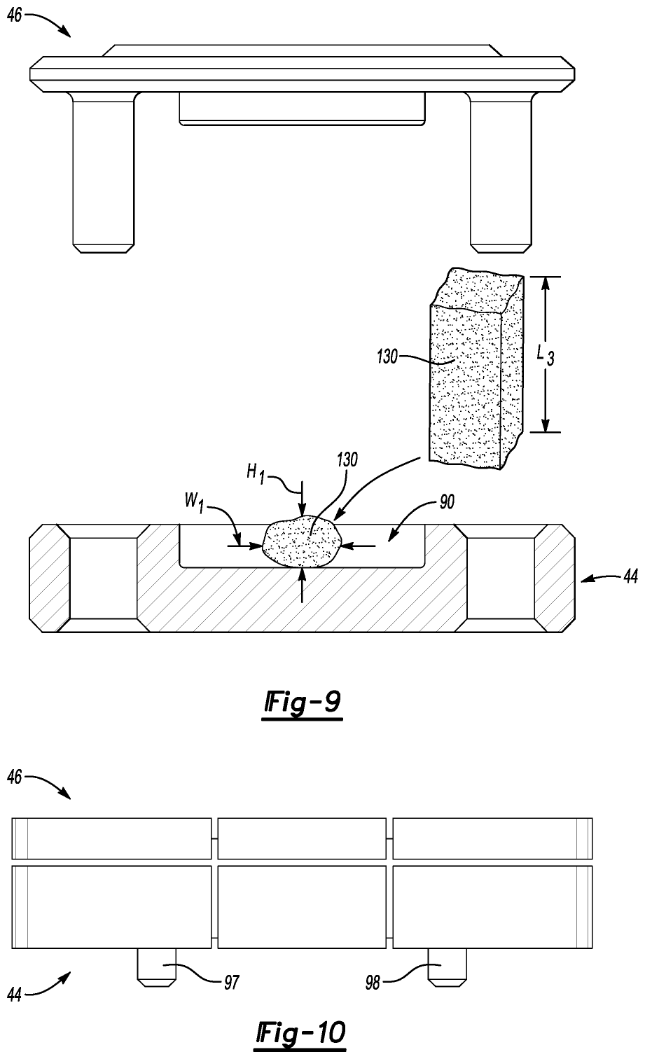

In the method, which is a surgical method, tissue is harvested from a first location in a body of a patient. In an example, the first location is one of the latissimus dorsi, pectoralis major, and fascia lata. This disclosure extends to other harvesting locations. Following harvesting, the example piece of harvested tissue 130, as shown in FIG. 9, initially exhibits a length L₁, width W₁, and a height H₁. The piece of harvested tissue 130 is placed within the cavity 90 of the first plate 44, as generally shown in FIG. 9.

Next, the second plate 46 is arranged relative to the first plate 44 such that the posts 97, 98 are received within one of the openings 94, 96. In a particular example, the posts 97, 98 project beyond a bottom surface of the first plate 44, as shown in FIG. 10. The combined first and second plates 44, 46 are then situated relative to the first press component 38. In particular, the combination of the first and second plates 44, 46 are lowered relative to the first press component 38 such that the portion of the posts 97, 98 projecting beyond the bottom of the first plate 44 are received in openings 62, 64. In this position, the first plate 44 is substantially within the channel 48. Further, the projections 66 are received in a corresponding one of the slots 102 of the first plate 44. In a particular example, the projections 66 project into the slots 102 such that top ends of the projections 66 lie in a plane containing the remainder of the cavity 90. In other words, the ends of the projections 66 are flush with the cavity 90. In this way, the harvested tissue is prevented from entering the slots 102 during compression.

Continuing with the example surgical method, the second press component 40 is then fit over the second plate 46 such that guide pins 58, 60 are received in a corresponding one of the openings 54, 56. Further, the raised section 78 is received in recessed section 124, and projections 80 are received in a corresponding one of the slots 122. In a particular example, the projections 80 project into the slots 122 such that the bottom ends of the projections 80 lie in a plane containing the remainder of the projection 120, which prevents the harvested tissue from entering the slots 122 during compression.

With the second press component 40 fit over the second plate 46, the assembly 36 of FIG. 2 is formed. The assembly 36 is then placed relative to the press 12. A compressive force is applied to the assembly 36 using the press 12. Specifically, with the first press component 38 resting on the base 22, rotation of the handle 34 results in downward movement of the connector 32, which is brought into contact with the second press component 40, and results in pressure being applied to assembly 36, which is transferred to the piece of harvested tissue 130. Eventually, the harvested tissue 130 is flattened and resembles FIG. 11, in which the harvested tissue 130 exhibits an height H₂ less than height H₁, and a width W₂ greater than width W₁.

The press 12 can release pressure from the assembly 36 by rotation of the handle 34 in an opposite direction. At this point, the plate assembly 42 can be removed from the first and second press components 38, 40. When removing the plate assembly 42 from the press 12 and from the remainder of the assembly 36, the interface between the posts 97, 98 and openings 94, 96 continues to hold first and second plates 44, 46 together such that the harvested tissue 130 is held under compression. In an example, the level of compression applied to the harvested tissue by the plates 44, 46 is less than the pressure applied by the press 12, but is sufficient to substantially maintain the flattened shape of the harvested tissue 130, at least for a period of time, while a user continues to work relative to the harvested tissue 130.

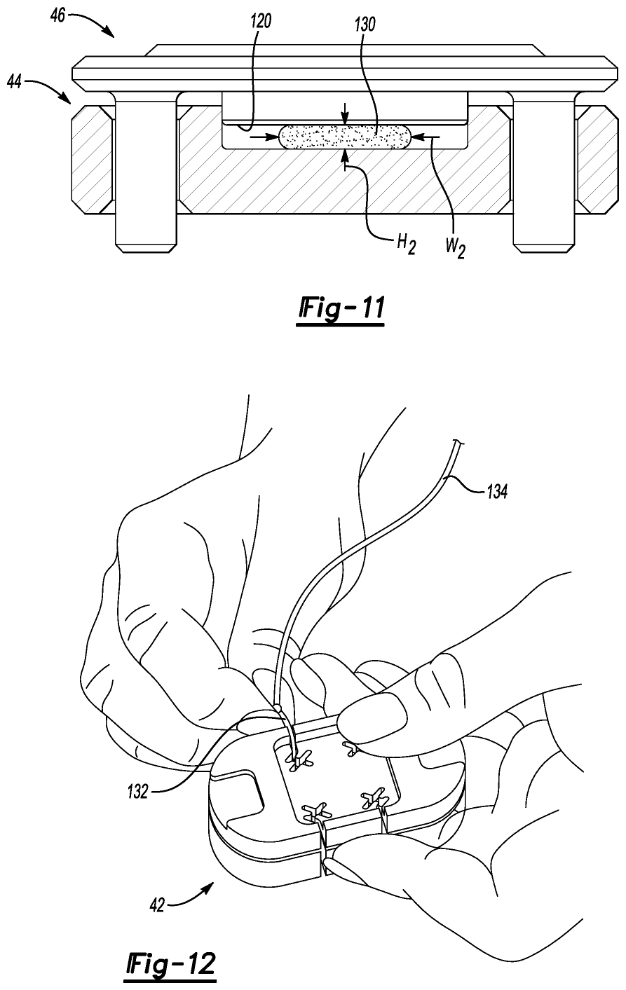

In a particular example, with the harvested tissue 130 still between the first and second plates 44, 46, a user may thread suture through the harvested tissue 130. In a particular example, a user may pass a needle 132 carrying suture 134 through one of the slots 122 in the second plate 46, through the harvested tissue 130, and through one of the slots 102 in the first plate 44, as generally represented in FIG. 12. The user may pass the needle 132 and suture 134 relative to the first and second plates 44, 46 and the harvested tissue 130 a number of times to create one or more stitches in the harvested tissue 130, including mattress stitches and/or luggage-tag stitches, as examples. Because the slots 102, 122 include sections (e.g., section 110) that open to the outer perimeter of a respective one of the first and second plates 44, 46, the ease of stitching the suture 134 relative to the harvested tissue 130 is increased and the suture 134 is readily separated from the first and second plates 44, 46. With the desired stitching complete, a user may separate the first and second plates 44, 46 by applying a force sufficient to overcome the friction created by the posts 97, 98 and openings 94, 96. The construct including the harvested tissue 130 and any stitches formed therein may then be removed from the cavity 90 and implanted in another location in a body of a patient.

In an example, the implanted location is adjacent a rotator cuff or another location. In this regard, the harvested tissue 130 is an autograft harvested from one location of a patient's body and used to repair or reconstruct a joint in another location of the patient's body. The harvested tissue 130 may be used to provide a partial or full replacement for a torn rotator cuff. This disclosure is not limited to use in rotator cuff repairs/reconstructions, however, and extends to the harvested tissue 130 being used in other repair or reconstruction techniques, including techniques involving repair or reconstruction of the hand, wrist, toe, Achilles tendon, Peroneal/Tibial tendon, among others. As another example, the harvested tissue 130 may be used in a superior capsular reinforcement technique.

In another aspect of the surgical method, before removing the assembly 36 from the press 12, one or more clamps may be attached to the plate assembly 42. Then, the assembly 36 can be removed from the press 12, and the plate assembly 42 can be separated from the first and second press component 38, 40 with the clamp(s) still clamping the first and second plates 44, 46 together. A user can thread suture relative to the harvested tissue 130 while the one or more clamps are attached to the first and second plates 44, 46.

In an aspect of this disclosure, the first press component 38, the second press component 40, the first plate 44, and the second plate 46 may be made of at least a semi-transparent material such that a harvested piece of tissue can be viewed through the first press component 38, the second press component 40, the first plate 44, and the second plate 46 while the press 12 is in use. In a further aspect of this disclosure the first press component 38, the second press component 40, the first plate 44, and the second plate 46 may be formed of a transparent material. An example transparent material is a clear polycarbonate material. Other transparent materials come within the scope of this disclosure. When the first press component 38, the second press component 40, the first plate 44, and the second plate 46 are made of semi-transparent or fully transparent material, the vertical dimension, or height, of the piece of harvested tissue may be more readily visible. A user of the press 12 can monitor the reaction of the piece of harvested tissue as compressive forces are applied to the piece of harvested tissue to determine if one or more dimensions of the harvested tissue have been changed as desired such that the user can cease use of the press 12. In other words, the user can determine whether the press 12 is applying an appropriate pressure to the harvested tissue for an appropriate time to achieve the desired change in dimension(s) of the harvested tissue.

It should be understood that directional terms such as top, bottom, upward, downward, etc., are used herein consistent with their art-accepted meaning and with reference to the normal operational orientation of the relevant components. These terms should not otherwise be considered limiting.

Although the different examples have the specific components shown in the illustrations, embodiments of this disclosure are not limited to those particular combinations. It is possible to use some of the components or features from one of the examples in combination with features or components from another one of the examples. In addition, the various figures accompanying this disclosure are not necessarily to scale, and some features may be exaggerated or minimized to show certain details of a particular component or arrangement.

One of ordinary skill in this art would understand that the above-described embodiments are exemplary and non-limiting. That is, modifications of this disclosure would come within the scope of the claims. Accordingly, the following claims should be studied to determine their true scope and content.

1. A system, comprising:
a press; and
a plate assembly including a first plate defining a cavity configured to receive harvested tissue and a second plate including a projection insertable into the cavity, wherein the press is configured to apply pressure to the plate assembly to compress the harvested tissue, and wherein the plate assembly is configured to hold the harvested tissue under compression when the plate assembly is removed from the press.

2. The system as recited in claim 1, wherein the second plate includes a post and the first plate includes an opening configured to receive the post.

3. The system as recited in claim 2, wherein the first plate includes an elastomeric liner within the opening and configured to directly contact the post.

4. The system as recited in claim 2, wherein:
the post is a first post and the second plate includes a second post,
the opening is a first opening and the first plate includes a second opening, and
the second opening is configured to receive the second post.

5. The system as recited in claim 4, wherein the first and second openings are through holes, and wherein the first and second posts are configured to project through the first plate.

6. The system as recited in claim 1, wherein the first plate includes a first slot and the second plate includes a second slot shaped substantially the same as first slot and configured to vertically overlap with the first slot.

7. The system as recited in claim 6, wherein the first slot and the second slot each include a first section open to an outer perimeter of a respective one of the first and second plates.

8. The system as recited in claim 7, wherein the first slot and the second slot each include a second section and a third section intersecting with the second section.

9. The system as recited in claim 8, wherein the first slot and the second slot each include a fourth section connecting the first section to the third section.

10. The system as recited in claim 6, wherein:
the first slot is one of four first slots of the first plate, and
the second slot is one of four second slots of the second plate.

11. The system as recited in claim 6, wherein:
the press includes a first press component configured to contact the first plate and a second press component configured to contact the second plate,
the first press component includes a projection configured to fit within the first slot, and
the second press component includes a projection configured to fit within the second slot.

12. The system as recited in claim 10, wherein the projection of the first press component is sized such that, when the projection of the first press component is within the first slot, an end surface of the projection of the first press component lies in a plane containing the cavity of the first plate.

13. The system as recited in claim 10, wherein the projection of the second press component is sized such that, when the projection of the second press component is within second slot, an end surface of the projection of the second press component lies in a plane containing the projection of the second plate.

14. The system as recited in claim 11, wherein:
the first press component includes a channel and an opening adjacent the channel, and
the channel is configured to receive the first plate.

15. The system as recited in claim 14, wherein:
the second press component includes a raised section and a guide pin adjacent the raised section,
the raised section is configured to fit within a recess of the second plate, and
the guide pin is configured to fit within the opening of the first press component.

16. The system as recited in claim 1, wherein:
the first plate includes a recessed section,
the second plate includes a recessed section, and
the system further comprises a clamp configured to interface with the recessed sections of the first plate and second plate to hold the harvested tissue under compression.

17. A surgical method, comprising:
placing harvested tissue into a cavity of a first plate;
arranging a second plate relative to the first plate such that a projection of the second plate is configured to fit within the cavity of the first plate;
arranging the first plate, second plate, and harvested tissue relative to a press;
changing a dimension of the harvested tissue by using the press to apply pressure to the first and second plate; and
removing the first plate, second plate, and harvested tissue from the press with the first and second plate held together such that the first and second plate hold the harvested tissue under compression.

18. The surgical method as recited in claim 17, wherein, during the removing step, the first and second plate are held together at least in part by an interface between a post of the second plate received in an opening of the first plate.

19. The surgical method as recited in claim 17, further comprising:
after the removing step, and while the first and second plate hold the harvested tissue under compression, passing suture through the harvested tissue by passing suture through a slot of the first plate and a slot of the second plate.

20. The surgical method as recited in claim 17, further comprising:
before the removing step, using a clamp to hold a relative position of the first and second plates.
